(19) United States
(12) Patent Application Publication
(10) Pub. No.: US 2003/0180241 A1
(43) Pub. Date: Sep. 25, 2003

(54) Deodorant composition for permanent wave

(75) Inventors: Kazutoshi Sakurai, Hiratsuka-shi (JP); Masataka Miyasaka, Hiratsuka-shi (JP); Junko Nagano, Hiratsuka-shi (JP)

Correspondence Address: SUGHRUE MION, PLLC, 2100 PENNSYLVANIA AVENUE, N.W., WASHINGTON, DC, 20037, US

(73) Assignee: TAKASAGO INTERNATIONAL CORPORATION

(21) Appl. No.: 10/390,652

(22) Filed: Mar. 19, 2003

(30) Foreign Application Priority Data
Mar. 22, 2002 (JP) P. 2002-80168

(51) Int. Cl. A61K 7/09; A61K 7/00

(52) U.S. Cl. 424/70.2

(57) ABSTRACT

A deodorant composition for permanent wave, which is excellent in stability and has a superior effect in masking unpleasant smell originated from mercapto compounds not only during the permanent treatment but also after the treatment. A deodorant composition is prepared by containing at least one compound or essential oil selected from Koavone, linalool oxide, rosephenone, Sandalore, Sandalmysore core, Bacdanol, Kephalis, cis-p-menthan-7-ol, nerolidol, raspberry ketone, α,3,3-trimethylcyclohexanemethyl formate, p-methoxyphenethyl alcohol, 2,2,6-trimethylcyclohexanecarboxylic acid ethyl ester, 2,2,6-trimethyl-1-crotonylcyclohexane, tansy oil and basil oil.

### FIELD OF THE INVENTION

This invention relates to a deodorant composition for permanent wave, which has a superior effect to mask unpleasant smells originated from thioglycollic acid used in the wave treatment with permanent wave, ammonia used in pH adjusting agents and the like, also has a superior effect to mask unpleasant smell originated from mercapto compounds remaining during permanent wave treatment and also after the treatment and is excellent in storage stability.

### BACKGROUND OF THE INVENTION

Conventionally, wave treatment of hair by permanent wave preparations is carried out using a first preparation containing a reducing agent and the like (to be referred sometimes to as “permanent wave first preparation” hereinafter) and a second preparation containing an oxidizing agent and the like (to be referred sometimes to as “permanent wave second preparation” hereinafter). Since reducing agent in the first preparation contains thioglycollic acid or the like mercapto compound as the main component, permanent wave treatment accompanies an unpleasant smell, and this unpleasant smell remains on hair and cannot be removed easily by shampooing in many cases.

Several methods for removing this unpleasant smell caused by permanent wave have been reported. For example, deodorant compositions for masking unpleasant smell originated from mercapto compounds and the like by containing a specified plant extract in permanent wave preparations, and a natural essential oil therein, have been reported in JP-A-4-247013 and JP-A-8-277210, respectively.

However, since the first preparation of permanent wave preparations contains thioglycollic or the like reducing agent but also contains a large blending amount of ammonia which results in an alkalinity of from pH 8 to 11, it is the present situation that there are no plant essential oils and the like containing various aromatic components, which are chemically stable without causing changes in odor and also can sufficiently satisfy masking of unpleasant smell remained on hair after the treatment, so that further improvements are in demand.

Regarding this point, a deodorant composition containing one or more species selected from specified 6 fragrances has been reported in JP-A-2000-344636, stating that the unpleasant smell by permanent wave treatment can be removed.

Though these 6 fragrances show a reasonable effect, concern has been directed toward a fragrance which is further stable and has more higher masking effect.

On the other hand, the second preparation contains an oxidizing agent and is adjusted to an acidic pH value of approximately from 3 to 4 in most cases, and a fragrance is rarely used therein in general.

However, since hair is generally treated with the first preparation and then the hair is treated with second preparation in carrying out permanent wave of hair, not only masking in products of the first preparation or second preparation but also the masking effect during hair treatment and the masking effect after the hair treatment become important points.

Accordingly, concern has been directed toward the advent of a fragrance which shows stabilizing effect in the acidic second preparation and has sufficient effect to mask smells originated from thioglycollic acid and ammonia when hair are treated with the first preparation and second preparation, as a common means at the time of permanent wave treatment, or the smells originated from other compounds, e.g., alkanolamines, sulfite and the like which are used as base material-constituting components.

### SUMMARY OF THE INVENTION

An object of the present invention is to provide a deodorant composition for permanent wave, which has excellent stability, can mask not only the unpleasant smells originated from mercapto compounds, ammonia and the like in the permanent wave first preparation but also the unpleasant smells originated from other compounds, e.g., alkanolamines, sulfite and the like which are used as base material-constituting components, can be formulated in the second preparation, shows excellent masking effect not only during the permanent wave treatment but also after the treatment, and can select any aroma which suits consumer's preference.

With the aim of solving above problems, the present inventors have conducted intensive studies and found as a result that a specified fragrance is stable even in alkaline and acidic solutions, has excellent masking effect and retains a desirable odor for a while after permanent wave treatment, and have reached the present invention by further continuing the studies.

Accordingly, the present invention includes a deodorant composition for permanent wave, which comprises a composition containing at least one compound or essential oil selected from acetyl diisoamylene, linalool oxide, rosephenone, 2-methyl-4-(2,2,3-trimethyl-3-cyclopenten-1-yl)-2-buten-1-ol, 3-methyl-5-(2,2,3-trimethyl-3-cyclopenten-1-yl)-pentan-2-ol, 2-ethyl-4-(2,2,3-trimethyl-3-cyclopenten-1-yl)-2-buten-1-ol, 4-(1-ethoxyvinyl)-3,3,5,5-tetramethylcyclohexanone, cis-p-menthan-7-ol, nerolidol, raspberry ketone, α,3,3-trimethylcyclohexanemethyl formate, p-methoxyphenethyl alcohol, 2,2,6-trimethylcyclohexanecarboxylic acid ethyl ester, 2,2,6-trimethyl-1-crotonylcyclohexane, tansy oil and basil oil, and

a composition for permanent wave, which contains the deodorant composition.

### DETAILED DESCRIPTION OF THE INVENTION

The following describes the present invention in detail.

The specified fragrances to be used in the deodorant of the present invention are already known and can be easily obtained by purchasing commercial products. A part of the fragrances can also be synthesized.

That is, acetyl diisoamylene (Koavone, a trade name of IFF), linalool oxide, rosephenone, 2-methyl-4-(2,2,3-trimethyl-3-cyclopenten-1-yl)-2-buten-1-ol (Sandalmysore core, a trade name of Kao Corp.), 3-methyl-5-(2,2,3-trimethyl-3-cyclopenten-1-yl)-pentan-2-ol (Sandalore, a trade name of Givaudan), 2-ethyl-4-(2,2,3-trimethyl-3-cyclopenten-1-yl)-2-buten-1-ol (Bacdanol, a trade name of IFF), 4-(1-ethoxyvinyl)-3,3,5,5-tetramethylcyclohexanone (Kephalis, a trade name of Givaudan), cis-p-menthan-7-ol (Mayol, a trade name of Firmenich), nerolidol, raspberry ketone, α,3,3-trimethylcyclohexanemethyl formate (Apharmate, a trade name of IFF), p-methoxyphenethyl alcohol, 2,2,6-trimethylcyclohexanecarboxylic acid ethyl ester (Thesaron, a trade name of Takasago International Corporation), 2,2,6-trimethyl-1-crotonylcyclohexane (Dihydrodamascone) and the like to be used in the deodorant of the present invention can be easily obtained by purchasing commercial products. Also, linalool oxide, rosephenone, nerolidol, raspberry ketone and the like may be prepared personally. In addition, tansy oil, basil oil and the like can also be easily obtained by purchasing commercial products. These fragrances may be racemic compounds or optically active substances in response to uses.

These fragrances show excellent effect in masking unpleasant smells caused by thioglycollic acid and ammonia and its derivatives formulated in permanent wave first preparation and the like and are also excellent in view of aromatic stability and chemical stability.

Particularly in Thesaron and Dihydrodamascone among these fragrances, the presence of stereoisomers (cis and trans) for the cyclohexane ring is known in view of their chemical structures, and the presence of many optically active substances having different optical activities is also known due to the presence of asymmetric carbon atoms. These stereoisomers and optically active substances are slightly different in terms of aroma and stability, but their aroma and stability are generally excellent in comparison with other fragrances. Thus, since their stereoisomers and optically active substances can be optionally selected in response to their use, a racemic body mixture may be used as such or a commercially available optically active substance may be used after all according to the present invention.

Further desirable results can be obtained when two or more of the compounds or essential oils defined by the present invention are jointly used. For example, a composition containing a compound or essential oil as a component A selected from acetyl diisoamylene, linalool oxide, rosephenone, 2-methyl-4-(2,2,3-trimethyl-3-cyclopenten-1-yl)-2-buten-1-ol, 3-methyl-S-(2,2,3-trimethyl-3-cyclopenten-1-yl)-pentan-2-ol, 2-ethyl-4-(2,2,3-trimethyl-3-cyclopenten-1-yl)-2-buten-1-ol, 4-(1-ethoxyvinyl)-3,3,5,5-tetramethylcyclohexanone, cis-p-menthan-7-ol, nerolidol, raspberry ketone, α,3,3-trimethylcyclohexanemethyl formate, p-methoxyphenethyl alcohol, tansy oil and basil oil, and a compound as a component B selected from 2,2,6-trimethylcyclohexanecarboxylic acid ethyl ester (Thesaron) and 2,2,6-trimethyl-1-crotonylcyclohexane (Dihydrodamascone) shows excellent effect in masking unpleasant smells caused by ammonia and its derivatives and is also excellent in view of aromatic stability and chemical stability.

Though blending ratio of these component A and component B is not particularly limited, it is desirable that the blending ratio of the component A and component B is set to, e.g., from 99.5:0.5 to 10:90 (weight ratio), more desirably from 95.5:5 to 20:80 (weight ratio).

In addition to the aforementioned fragrance components, other optional components can be formulated in the deodorant composition of the present invention in response to the object, kind of the composition and the like. As the components to be formulated, for example, other fragrance components than the aforementioned fragrances, diluents, solvents and the like can be cited. Examples of the other fragrance components include p-cymene, terpinolene, myrcene and the like terpene system hydrocarbons, heptanal, octanal, benzaldehyde, salicylic aldehyde, citronellal, alpha-hexylcinnamic aldehyde and the like aldehydes, methyl jasmonate, gamma-nonalactone, gamma-decalactone, coumarin and the like esters, anisole, paracresyl methyl ether, beta-naphthol methyl ether, beta-naphthol ethyl ether and the like ethers, and menthone, acetophenone, alpha-damascone, beta-damascone, α-ionone, β-ionone, methylionone, irone, dihydrojasmone and the like ketones. Also included are isopropyl alcohol, cis-3-hexenol, heptanol, 2-octanol, benzyl alcohol, citronellol, geraniol, terpineol, tetrahydrogeraniol, anise alcohol, phenethyl alcohol and the like alcohols.

Also, as the diluent and solvent, dipropylene glycol, ethanol, 3-methoxy-3-propanol and the like are desirable from the general purpose point of view.

It is possible to formulate an ultraviolet ray absorbent, a hair protecting agent and the like as other effective components in the deodorant composition of the present invention.

The deodorant composition for permanent wave of the present invention can be used by formulating in the permanent wave first preparation and permanent wave second preparation, and shampoo, hair rinse, hair treatment, hair cream, hair lotion, hair foam and the like hair care products which are used before the permanent wave treatment or after the permanent wave treatment.

The deodorant composition of the invention is blended at an amount of preferably 0.01 to 30% by weight, particularly preferably 0.1 to 1.0% by weight based on the total amount of the permanent wave first preparation. Further, the same is true with the permanent wave second preparation.

The deodorant composition of the invention may be blended at an amount of 0.01 to 30% by weight, particularly 0.1 to 2.0% by weight to the total amount of a hair treatment composition. For example, the deodorant composition of the invention may be blended at an amount of 0.01 to 30% by weight, particularly 0.1 to 2.0% by weight based on the total amount of a first agent and a second agent. In that case, the deodorant composition of the invention may satisfactorily be blended in either one or both of the first agent and the second agent.

A reducing agent is added to the deodorant composition of the invention, to prepare a permanent wave composition, specifically a permanent wave first preparation.

An oxidizing agent is added to the deodorant composition of the invention, to prepare a permanent wave composition, specifically a permanent wave second preparation.

Such two agent-type permanent wave composition, which contain these first preparation and second preparation, are also the inventive products.

As the reducing agent to be blended in the permanent wave first preparation of the invention, for example, thioglycolic acid, tioglycolates such as ammonium tioglycolate, dithioglycolic acid, thioglycerol, cysteine, salts of cysteine, and acetyl cysteine. The content of the reducing agent in the permanent wave first preparation is not particularly limited and is preferably from 3 to 25% by weight, particularly preferably 5 to 20% by weight based on the total amount of such first preparation.

The permanent wave first preparation may further contain an alkali agent. Examples of the alkali agent to be blended include ammonia, aqueous ammonia, monoethanolamine, triethanolamine, isopropanolamine, ammonium hydrogencarbonate, potassium hydroxide, sodium hydroxide, and the like. The content of the reducing agent in the permanent wave first preparation is not particularly limited and is preferably from 0.1 to 15% by weight, particularly preferably 0.2 to 10% by weight based on the total amount of such first preparation.

Typically, the first preparation is adjusted to preferably pH 8 to 12, particularly preferably pH 8 to 11.

Examples of the oxidizing agent to be contained in the permanent wave second preparation includes hydrogen peroxide, urea peroxide, sodium bromate, potassium bromate, sodium peroxobrorate, and the like. The content thereof is not particularly limited and is preferably from 1 to 20% by weight, particularly preferably from 5 to 15% by weight based on the total amount of the second preparation.

Typically, the second preparation is adjusted to preferably pH 2 to 5, particularly preferably pH 2 to 4.

The permanent wave composition in accordance with the invention may appropriately be blended with other additives such as nonionic surfactants; anionic surfactants such as alkyl sulfate ester salt and polyoxyethylene alkyl ether sulfate salt; cationic surfactants; ampholytic surfactants such as alkyldimethylaminoacetate betaine; moisturizers such as glycerin and propylene glycol; oily ingredients; stabilizers; thickeners such as carboxymethyl cellulose, hydroxyethyl cellulose, and hydroxypropyl cellulose; pH adjusters such as phosphoric acid, hydrochloric acid, phosphate dihydrogen monopotassium, citric acid, succinic acid and sodium hydroxide; oil agent such as lanolin, cetanol, cholesterol, liquid paraffin, petrolatum, squalane; chelating agents such as ethylenediaminetetraacetic acid; anti-oxidants; and preservatives.

For the permanent wave composition in accordance with the invention, further, media such as water and/or organic solvents may be used. The organic solvents include ethanol, benzyl alcohol, propylene glycol, diethylene glycol and glycerin.

The permanent wave composition in accordance with the invention can be formulated into, for example, liquid, cream, gel, paste, mousse, solid, aerosol, foam and powder.

The permanent wave treatment using the deodorant composition for permanent wave of the invention is carried out, for example, as follows. First, the permanent wave first preparation of the invention is applied to hair, the hair may be rolled with rods if necessary, and the hair is left as it is. Then, the permanent wave second preparation is applied to the hair, and the hair is left as it is. Subsequently, the hair is rinsed and dried. As occasion demands, the hair may be left with heating to from 40° C. to 60° C.

It is particularly desirable to use it by formulating in a hair treating preparation, such as the permanent wave first preparation, which contains ammonia for pH adjustment jointly with thioglycollic acid, because it can mask not only the unpleasant smell originated from ammonia released from the products but also the unpleasant smell from the hair during and after the permanent wave treatment.

The following describes the present invention further in detail with reference to examples, but the present invention is not restricted by these examples.

### EXAMPLE 1

A first preparation for permanent wave was prepared based on the formulation shown in Table 1. In this connection, illustrative example of the deodorant composition in Table 1 is shown in Table 2.

TABLE 1

| Compounds | Weight % |
|---|---|
| Disodium edetate | 0.5 |
| Aqueous ammonia (28%) | 2.0 |
| Ammonium thioglycolate (50%) | 13.5 |
| Stearyltrimethylamonium chloride aqueous | 35 |
| solution | |
| Ammonium bicarbonate | 2.5 |
| Citric acid | 0.5 |
| Diethanolamide laurate | 14.5 |
| Polyoxyethylene sorbitan monolaurate | 1.0 |
| (Leodol TW-L 120) | |
| Propylene glycol | 1.0 |
| Deodorant composition | 0.2 |
| purified water | balance |

(Evaluation) Effect of the aforementioned first preparation for permanent wave to mask the unpleasant smell originated from ammoniacal smell and stability of its aroma were evaluated by the following evaluation method.

(Evaluation Method)

(1) Masking Effect;

Each permanent wave preparation was stored at 43° C. for 12 weeks, and then its unpleasant smell-masking effect was evaluated by sensory test based on the following criteria.

A; unpleasant smell is not felt

B; unpleasant smell is hardly felt

C; unpleasant smell is slightly felt

D; unpleasant smell is felt

(2) Stability of Aroma and Masking Effect;

A permanent wave treatment was carried out by a conventional method using the aforementioned permanent wave first preparation. (2-a) Stability effect: Changing degree of the aromatic tone at the bottle mouth when the container was opened before the treatment was evaluated by sensory test based on the following criteria.

A; no change, B; slightly changed, C; fairly changed, D; greatly changed

(2-b) Masking effect: A smell from hair during the treatment (just after application to hair) and after the treatment was evaluated by sensory test based on the following criteria.

A; very good, B; slightly good, C; usual or slightly bad, D; bad

Results of the evaluation by the above evaluation methods are shown in Table 2. In this connection, the “masking effect (1)” in the table is an evaluation result by the above evaluation method (1), the “stability of aroma” is an evaluation result by the above evaluation method (2-a), and other “masking effect” is an evaluation result by the above evaluation method (2-b).

TABLE 2

| Deodorant | Masking | Stability | Masking effect | |
|---|---|---|---|---|
| composition | Effect | of | during | after |
| (fragrance 100%) | (1) | aroma | treatment | treatment |
| Koavone (IFF) | A | A | A | A |
| Linalool oxide | A | A | A | A |
| Rosephenone | A | A | A | A |
| Sandalmysore core | A | A | A | A |
| (Kao) | | | | |
| Sandalore | A | A | A | A |
| (Givaudan) | | | | |
| Bacdanol (IFF) | A | A | A | A |
| Levosandol | A | A | A | A |
| (Takasago) | | | | |
| Kephalis (Givaudan) | A | A | A | A |
| Mayol (Firmenich) | A | A | A | A |
| Nerolidol | A | A | A | A |
| Thesaron (Takasago) | A | A | A | A |
| Dihydrodamascone | A | A | A | A |
| Raspberry ketone | A | A | A | A |
| Apharmate | A | A | A | A |
| (Firmenich) | | | | |
| p-Methoxyphenethyl | A | A | A | A |
| alcohol | | | | |
| Tansy oil | A | A | A | A |
| Basil oil | A | A | A | A |

levosandol: (E)-(R)-2-ethyl-4-(2,2,3-trimethyl-3-cyclopenten-1-yl)-2-buten-1-ol

As is evident from the results of Table 2, it was revealed that all preparations of the present invention are excellent in terms of stability, have high masking effect and exert superior effects even during the hair treatment or after the treatment.

### EXAMPLE 2

A first preparation for permanent wave composition containing the deodorant composition shown in Table 3 was obtained based on the formulation shown in Table 1.

TABLE 3

| Deodorant composition | Weight |
|---|---|
| (mixed fragrances) | % |
| Levosandol (Takasago) | 2.0 |
| Rosephenone | 10.0 |
| Linalool oxide | 2.0 |
| Kephalis (Givaudan) | 5.0 |
| Nerolidol | 8.0 |
| Raspberry ketone | 0.5 |
| Apharmate (Firmenich) | 2.5 |
| p-Methoxyphenethyl alcohol | 20.0 |
| Tansy oil | 5.0 |
| Mayol (Firmenich) | 10.0 |
| Methyl ionone | 5.0 |
| Menthone | 1.0 |
| Rose oxide | 1.0 |
| Tetrahydrolinalool | 10.0 |
| Musk T (Takasago) | 5.0 |
| Thesaron (Takasago) | 5.0 |
| Dihydrodamascone | 1.0 |
| Ethyl-2-methyl butyrate | 1.5 |
| Dipropylene glycol | 5.5 |

(Evaluation) Effect of the aforementioned permanent wave first preparation to mask the unpleasant smell originated from ammoniacal smell and stability of its aroma were evaluated in the same manner as in Example 1. That is, (1) masking effect on the unpleasant smell originated from ammoniacal smell, (2-a) stability effect when a permanent wave treatment was carried out by a conventional method using the permanent wave first preparation and (2-b) masking effect when a permanent wave treatment was carried out by a conventional method using the permanent wave first preparation were evaluated in the same manner as in Example 1.

The results are shown in Table 4.

TABLE 4

| | (1) | (2-a) | (2-b) |
|---|---|---|---|
| Fragrance mixture | A | A | A |

As is evident from the results shown in Table 4, all of the products of the present invention were excellent in stability and has high masking effect. Particularly in (2-b), it was valued that aroma from hair during the permanent wave treatment (just after application to hair) and after the treatment was vary good in both cases when a permanent wave treatment was carried out by a conventional method using the permanent wave first preparation.

### EXAMPLE 3

A second preparation composition for permanent wave was prepared by the formulation shown in Table 5. In this connection, illustrative example of the deodorant composition in Table 5 are shown in Table 6.

TABLE 5

| Compound | Weight % |
|---|---|
| Sodium bromate | 8.0 |
| Polyoxyethylene lauryl ether sodium | 5.0 |
| sulfate | |
| (2.5 E.O.) (Leodol TW-L 120) | |
| Polyoxyethylene tridecyl ether | 1.0 |
| 48% Sodium hydroxide aqueous solution | proper amount |
| (pH adjusting agent) | |
| Isopropyl palmitate | 2.0 |
| Citric acid (pH adjusting agent) | proper amount |
| Anhydrous sodium sulfate | 1.0 |
| Deodorant composition | 0.2 |
| Purified water | balance |

(Evaluation) Masking effect of the aforementioned second preparation for permanent wave and stability of its aroma were evaluated by the following evaluation method.

(Evaluation Method)

(3) The aforementioned permanent wave second preparation was stored at 43° C. for 12 weeks, and then its masking effect was evaluated by a sensory test based on the following criteria.

A; unpleasant smell is not felt

B; unpleasant smell is hardly felt

C; unpleasant smell is slightly felt

D; unpleasant smell is felt

(4) Stability of Aroma and Masking Effect

A permanent wave treatment was carried out by a conventional method using the aforementioned permanent wave second preparation.

(4-a) Stability effect: Changing degree of the aromatic tone at the bottle mouth when the container containing each permanent wave second preparation was opened was evaluated by a sensory test based on the following criteria.

A; no change, B; slightly changed, C; fairly changed, D; greatly changed

(4-b) Masking effect: A permanent wave treatment was carried out by a conventional method using the permanent wave first preparation. One hour thereafter, treatment with each permanent wave second preparation was carried out by a conventional method. A smell from dry hair during the treatment (just after application to hair) and after the treatment (residual small) was evaluated by a sensory test based on the following criteria.

A; very good, B; slightly good, C; usual or slightly bad, D; bad

Results of the evaluation by the evaluation methods are shown in Table 6. In this connection, the “masking effect (3)” in the table is an evaluation result by the above evaluation method (3), the “stability of aroma” is an evaluation result by the above evaluation method (4-a), and other “masking effect” is an evaluation result by the above evaluation method (4-b).

TABLE 6

| | | | Masking effect | |
|---|---|---|---|---|
| | Masking | Stability | during | after |
| Compound | Effect | of aroma | treatment | treatment |
| Koavone (IFF) | A | A | A | A |
| Linalool oxide | A | A | A | A |
| Rosephenone | A | A | A | A |
| Sandalmysore core | A | A | A | A |
| (Kao) | | | | |
| Sandalore | A | A | A | A |
| (Givaudan) | | | | |
| Bacdanol (IFF) | A | A | A | A |
| Levosandol | A | A | A | A |
| (Takasago) | | | | |
| Kephalis (Givaudan) | A | A | A | A |
| Mayol (Firmenich) | A | A | A | A |
| Nerolidol | A | A | A | A |
| Raspberry ketone | A | A | A | A |
| Apharmate (IFF) | A | A | A | A |
| p-Methoxyphenethyl | A | A | A | A |
| alcohol | | | | |
| Tansy oil | A | A | A | A |
| Basiloil | A | A | A | A |

As shown in Table 6, the deodorant composition of the present invention was highly stable even in the permanent wave second preparation containing an oxidizing agent, masked the ammoniacal smell and also showed high stability even after subsequent permanent wave treatment by a conventional method, showing no changes in aroma.

The deodorant composition for permanent wave of the present invention has a superior effect in masking the unpleasant smell originated from thioglycollic acid and its salts contained in the permanent wave first preparation and also in masking the smell of ammonia used as a pH adjusting agent, is excellent in terms of periodical stability for a prolonged period of time of 3 months at 45° C. with less changes in aroma, and also has a superior effect in masking the unpleasant smell originated from mercapto compounds such as thioglycollic acid even after treatment with the permanent wave second preparation.

While the invention has been described in detail and with reference to specific embodiments thereof, it will be apparent to one skilled in the art that various changes and modifications can be made therein without departing from the scope thereof.

This application is based on Japanese patent application No. 2002-080168 filed Mar. 22, 2002, the entire contents thereof being hereby incorporated by reference.

What is claimed is:

1. A deodorant composition for permanent wave, which comprises at least one compound or essential oil selected from acetyl diisoamylene, linalol oxide, rosephenone, 2-methyl-4-(2,2,3-trimethyl-3-cyclopenten-1-yl)-2-buten-1-ol, 3-methyl-S-(2,2,3-trimethyl-3-cyclopenten-1-yl)-pentan-2-ol, 2-ethyl-4-(2,2,3-trimethyl-3-cyclopenten-1-yl)-2-buten-1-ol, 4-(l-ethoxyvinyl)-3,3,5,5-tetramethylcyclohexanone, cis-p-menthan-7-ol, nerolidol, raspberry ketone, α,3,3-trimethylcyclohexanemethyl formate, p-methoxyphenethyl alcohol, 2,2,6-trimethylcyclohexanecarboxylic acid ethyl ester, 2,2,6-trimethyl-1-crotonylcyclohexane, tansy oil and basil oil.

2. A deodorant composition for permanent wave, which comprises
at least one compound or essential oil selected from acetyl diisoamylene, linalol oxide, rosephenone, 2-methyl-4-(2,2,3-trimethyl-3-cyclopenten-1-yl)-2-buten-1-ol, 3-methyl-5-(2,2,3-trimethyl-3-cyclopenten-1-yl)-pentan-2-ol, 2-ethyl-4-(2,2,3-trimethyl-3-cyclopenten-1-yl)-2-buten-1-ol, 4-(1-ethoxyvinyl)-3,3,5,5-tetramethylcyclohexanone, cis-p-menthan-7-ol, nerolidol, raspberry ketone, α,3,3-trimethylcyclohexanemethyl formate, p-methoxyphenethyl alcohol, tansy oil and basil oil as component A, and
at least one compound selected from 2,2,6-trimethylcyclohexanecarboxylic acid ethyl ester and 2,2,6-trimethyl-1-crotonylcyclohexane as component B.

3. A composition for permanent wave comprising a deodorant composition according to claim 1 and a reducing agent.

4. A composition for permanent wave comprising a deodorant composition according to claim 2 and a reducing agent.

5. A composition for permanent wave comprising a deodorant composition according to claim 1 and an oxidizing agent.

6. A composition for permanent wave comprising a deodorant composition according to claim 2 and an oxidizing agent.

7. A composition for permanent wave comprising:
a first agent comprising a reducing agent and
a second agent comprising an oxidizing agent,
wherein either one or both of the first agent and the second agent comprises a deodorant composition according to claim 1.

8. A composition for permanent wave comprising:
a first agent comprising a reducing agent and
a second agent comprising an oxidizing agent,
wherein either one or both of the first agent and the second agent comprises a deodorant composition according to claim 2.

9. A method for deodorizing the odor during and after permanent treatment, using a deodorant composition according to claim 1.

10. A method for deodorizing the odor during and after permanent treatment, using a deodorant composition according to claim 2.
